(19) United States
(12) Patent Application Publication
(10) Pub. No.: US 2005/0033293 A1
(43) Pub. Date: Feb. 10, 2005

(54) Transtrochanteric rotational osteotomy

(76) Inventor: Taek-Rim Yoon, Kwangju-city (KR)

Correspondence Address: MERCHANT & GOULD PC, P.O. BOX 2903, MINNEAPOLIS, MN, 55402-0903, US

(21) Appl. No.: 10/629,960

(22) Filed: Jul. 30, 2003

(51) Int. Cl. A61F 2/30

(52) U.S. Cl. 606/60

(57) ABSTRACT

This invention is a surgical method to treat a disease called osteonecrosis (Avascular necrosis, Aseptic necrosis) of the femoral head which is caused by interruption of blood supply to the femoral head. This method comprises several surgical techniques including circular incision of hip joint capsule, osteotomy through trochanteric area of the femur without detaching the greater trochanter, rotation of the femoral head and neck portion and internal fixation with screws. The purpose of these procedures is to make the femoral head rotated so that the intact portion of the femoral head can support the pelvis and the necrotic portion of the femoral head can be placed in the non-weight bearing portion.

### FIELD OF THE INVENTION

The present invention relates to a surgical method of treating a disease called osteonecrosis of the femoral head which is caused by interruption of blood supply to the femoral head. This method comprises several surgical techniques including incision of hip joint capsule, osteotomy through trochanteric area of the femur without detaching the greater trochanter, rotation of the femoral head and neck portion and internal fixation with screws. In this procedure, the femoral head is rotated so that the necrotic portion of the femoral head can be placed in the non-weight bearing portion and the intact portion of the femoral head can be turned into superior portion to support the pelvis.

### DESCRIPTION OF THE PROR ART

Osteonecrosis of the femoral head (1) is a disease coming from an interruption of the blood supply to the femoral head. This condition leads to the destruction of femoral head making the patients feel pain in the hip joint, limitation of the joint motion, limping and, if it is severe, inability to walk.

There have been many kinds of surgical methods to treat this disease including core decompression, multiple drilling, osteotomy, bone grafting, and hip replacement. The fact that there have been many methods to treat this disease implies that there has been no single best method. The present invention is a kind of osteotomy. The previous methods of osteotomy could be divided into two categories: proximal femoral varus osteotomy or flexion osteotomy and transtrochanteric rotational osteotomy. The present invention is a kind of the transtrochanteric rotational osteotomy (FIGS. 1, 2, 3). The previous transtrochanteric rotational osteotomy has disadvantages to osteotomize the greater trochanter(2) first and to re-attach the greater trochanter with cerclage wiring(5) at the end of the osteotomy and fixation. It takes more time of operation and has possibility of the nonunion of the greater trochanter.

Therefore, if the femoral head was destroyed despite many efforts to preserve the femoral head, there was no choice but to replace the hip with artificial joint.

### SUMMARY OF THE INVENTION

This invention is a surgical method to treat a disease called osteonecrosis of the femoral head which is caused by interruption of blood supply to the femoral head. In many of the patients, the avascular area occupies anterosuperior portion of the femoral head and the vascular area occupies posteroinferior portion of the femoral head. The avascular area are finally collapsed producing hip pain. Further development of the disease will lead to arthritis of the hip joint and final destruction of the joint. This method was developed to treat this disease by rotating the femoral head. This method comprises several surgical techniques including skin incision for exposure, incision of hip joint capsule, osteotomy through trochanteric area of the femur without detaching the greater trochanter, rotation of the femoral head and neck portion, internal fixation with screws and closure of the skin. The purpose of these procedures is to make the femoral head rotated so that the intact portion of the femoral head can support the pelvis and the necrotic portion of the femoral head can be placed in the non-weight bearing portion.

### BRIEF DESCRIPTION OF THE DRAWINGS

For fuller understanding of the nature and objects of the invention, reference should be made to the following detailed description taken in conjunction with the accompanying drawings in which:

FIG. 1 is a schematic diagram for illustrating the outline of the osteotomy of the conventional rotational transtrochanteric osteotomy. The greater trochanter is osteotomized first and another osteotomy is then performed perpendicular to the axis of the femoral neck

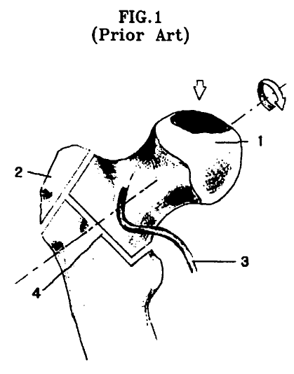

FIG. 2 is a schematic diagram for illustrating the proximal femur after rotation of the femoral head and neck portion.

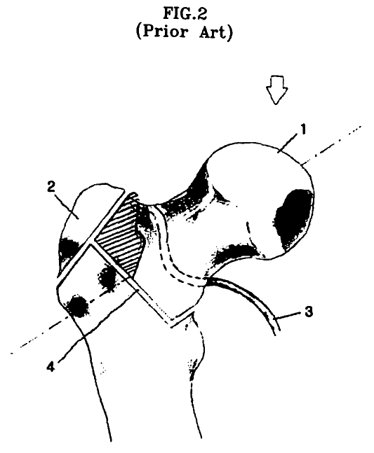

FIG. 3 is a schematic diagram for illustrating the fixation after the osteotomy The greater trochanter need also be fixed with cerclage wiring.

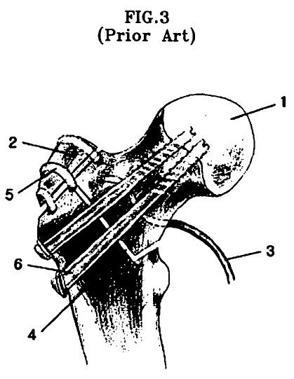

FIG. 4 is a schematic diagram for illustrating the outline of the new osteotomy. The osteotomy is performed without complete detachment of the greater trochanter.

FIG. 5 is a schematic diagram for illustrating the proximal femur rotation of the femoral head and neck portion.

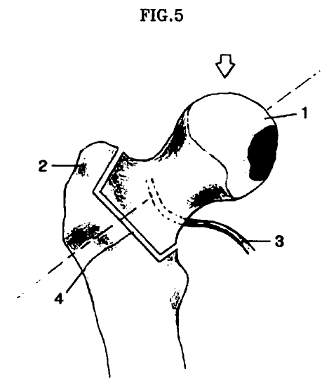

FIG. 6 is a schematic diagram for illustrating the fixation after the osteotomy and rotation of the femoral head and neck portion. There is no need for fixation of the greater trochanter.

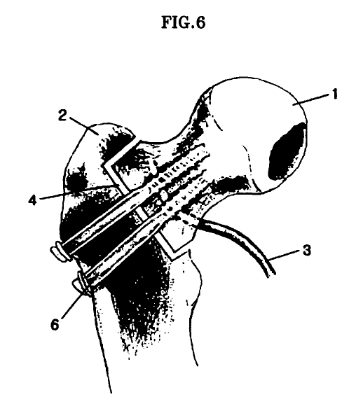

### DETAILED DESCRIPTION OF THE INVENTION

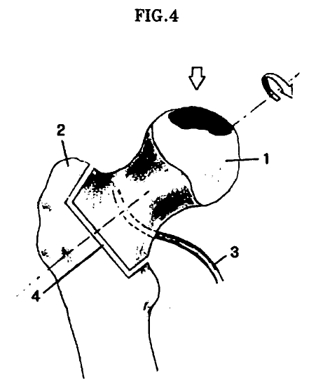

A preferred embodiment of the present invention will now be described in detail with reference to the accompanying drawings (FIG. 4, 5, 6). First of all, with the patient in the lateral decubitus position, a skin incision is made over the hip and the femur. The capsule of the hip joint is exposed through a lateral approach. The anterior side and posterior side of the hip are exposed. The short external rotators except Quadratus femoris muscle are completely transected. The posterior column branch of medial circumflex artery (3) which is just above the lesser trochanter must be preserved. The joint capsule is widely exposed anteriorly and posteriorly and is then incised circumferentially.

The line of osteotomy (4) is drawn as in FIG. 4 and K-wires are placed perpendicular to the neck, and X-ray is taken in the A-P plane to confirm the osteotomy line before the transtrochanteric osteotomy,

If the level of osteotomy site is confirmed, the site is then osteotomized with a saw. The osteotomy site is separated and any remaining joint capsule is incised because the remaining joint capsule may prevent the rotation of the femoral head and neck portion. If the amount of rotation is satisfactory, the osteotomy site is temporarily fixed with pins or screws. The position of the osteotomy site is confirmed by using x-rays or image intensifiers. If the position is satisfactory again, the osteotomy site is fixated with screws (6). The muscle fascia and subcutaneous tissue are repaired over a suction drain. The skin is closed.

Osteonecrosis of the femoral head is a disease coming from an interruption of the blood supply to the femoral head. This condition, if untreated well, leads to the destruction of femoral head making the patients feel pain in the hip joint, limitation of the joint motion, limping and, if it is severe, inability to walk, and finally leads to complete destruction of the joint and the joint needs to be replaced with an artificial joint.

This invention is a surgical method of treating osteonecrosis of the femoral head by rotating the femoral head so that the intact portion of the femoral head can be rotated into superior portion and to support pelvis without detaching the greater trochanter.

This invention has many advantages such as: no detachment of the greater trochanter, no fixation of the greater trochanter, less operation time, no anatomical disturbance of the greater trochanter, and early rehabilitation. With this new invention, the osteonecrosis of the femoral head can be treated much easily and successfully with minimal discomfort of the patients.

1. A surgical method to treat osteonecrosis of the femoral head comprising (1) a surgical step to osteotomize trochanteric area without detaching the greater trochanter, (2) a surgical step to rotate the head and neck portion so that the intact portion of the head can be rotated superiorly, and (3) a surgical step to fix the osteotomy site by screws.
